(19) United States
(12) Patent Application Publication
(10) Pub. No.: US 2026/0297501 A1
(43) Pub. Date: Oct. 1, 2026

(54) Agitators and Methods for Using and Manufacturing the Same

(71) Applicant: Sartorius Stedim North America Inc., Bohemia, NY (US)

(72) Inventors: Michael Zumbrum, New Oxford, PA (US); William Kimmick, Mechanicsburg, PA (US); Mark Szczypka, Bohemia, NY (US); David Splan, Bohemia, NY (US); Priya Anandakumaran, Bohemia, NY (US); Samin Akbari, Bohemia, NY (US)

(73) Assignee: Sartorius Stedim North America Inc., Bohemia, NY (US)

(21) Appl. No.: 19/093,802

(22) Filed: Mar. 28, 2025

(51) Int. Cl. C12N 1/02; B01L 3/00; C12M 1/00

(52) U.S. Cl. CPC C12N 1/02; B01L 3/502; C12M 47/02; B01L 2400/086

(57) ABSTRACT

An agitator includes a unitary body defining an inlet, an outlet, and a fluid channel extending between the inlet and the outlet. The fluid channel has a disruptor section with a plurality of chambers that are in fluid communication with one another. Each chamber creates a trituration point within the disruptor section.

### BACKGROUND

### 1. Technical Field

The present disclosure relates to agitators and, more specifically, to agitators formed of a single unitary body and methods of using and manufacturing the same.

### 2. Discussion of Related Art

Cells can be cultured for a variety of uses, for example, to manufacture biological products and pharmaceutical products. Cells tend of clump together and aggregate making them difficult to process. For example, some cells are cultured while attached to microcarriers such as beads. To harvest the cells, the cells are separated from the microcarriers for use.

Agitation of cellular suspensions has been used for many years with mechanical devices such as vortex mixers. Repeated drawing and discharging of fluids using pipettes is also a technique known to those skilled in the art of bioprocessing. One system is disclosed in U.S. Pat. No. 11,524,293 and includes several parts that are secured together. The parts have a long flow path and a low ovulation to length ratio. There is a continuing need for improved devices, methods, and systems for separating cells from microcarriers, and for providing suspensions of single cells.

### SUMMARY

This application is directed generally to devices and methods for de-agglomerating mixtures containing living cells. The devices disclosed herein gently agitate cells to separate, disrupt, or de-agglomerate the cells within the mixture. For example, the devices disclosed herein may be used to separate cells from support structures such as microcarriers. The devices disclosed herein may be fabricated using additive manufacturing to create interior geometries that would be difficult if not impossible to produce with conventional techniques such as molding or machining. Additive manufacturing of the devices disclosed herein may allow for customizations of sizes from submicron to centimeters.

In addition, the devices disclosed herein may be designed with significant geometric freedom compared to devices manufactured from traditional techniques. Further, the devices disclosed herein may be monolithic structures formed of a single unitary piece reducing the number of parts and/or failure points. Creating the devices disclosed herein may allow for the numerous interior geometries to agitate a mixture without the need for connections between structures. The monolithic nature of the devices disclosed herein may increase the robustness of the disclosed devices over existing devices. The devices disclosed herein may have a shorter fluid path with an increased number of ovulations per unit of length or an increased ovulation to length ratio over other devices. The devices disclosed herein may be modified for a particular mixture between production runs digitally. The devices disclosed herein may have textures or surface features on the internal fluid pathways.

In an aspect of the present disclosure, an agitator includes a unitary body defining an inlet, an outlet, and a fluid channel extending between the inlet and the outlet. The fluid channel has a disruptor section with a plurality of chambers that are in fluid communication with one another. Each chamber creates a trituration point within the disruptor section.

In aspects, the disruptor section has a minimum diameter and a maximum diameter. The minimum diameter may be in a range of 0.001 millimeters to 10 millimeters. A ratio between the maximum diameter and the minimum diameter may be in a range of 2:1 to 100:1. The unitary body may define an inlet chamber between the inlet and the fluid channel and an outlet chamber between the fluid channel and the outlet. The fluid channel may include a plurality of fluid subchannels with each fluid subchannel having a respective disruptor section. The plurality of fluid subchannels may include four fluid sub-channels.

In some aspects, the plurality of chambers includes a first chamber, a second chamber, and a third chamber which are disposed in order with one another such that fluid flows downstream from the first chamber directly into the second chamber and directly into the third chamber from the second chamber. The first chamber may have a first maximum diameter, the second chamber may have a second maximum diameter that is less than the first maximum diameter, and the third chamber may have a third maximum diameter that is greater than the second maximum diameter. The third maximum diameter may be greater than the first maximum diameter. The first chamber, the second chamber, and the third chamber may be coaxially aligned with one another.

In certain aspects, the plurality of chambers includes at least five chambers in fluid communication with one another such that fluid flows sequentially through each of the five chambers as it flows through the fluid channel. The disruptor section may include smooth transitions between the maximum diameters of the chambers.

In particular aspects, a longitudinal length of the body is defined linearly between the inlet and the outlet of the agitator. The disruptor section may wrap around the body such that a fluid length of the disruptor section is greater than the longitudinal length of the body. The flow length of the disruptor section may be greater than twice the longitudinal length of the body. The disruptor section may be defined by chamber walls. The chamber walls may have a textured surface to promote shear within all fluid passing through the disruptor section.

In aspects, the disruptor section has the average or mean diameter. The mean diameter may be in a range of 0.1 microns to 2 centimeters, 5 microns to 500 microns, or 0.1 centimeters to 0.5 centimeters. The disruptor section may be configured to deagglomerate cells from one another within a fluid as the fluid flows through the disruptor section. The disruptor section may be configured to separate cells from microcarriers as the fluid flows through the disruptor section.

In another aspect of the present disclosure, an agitator system includes an agitator, a first aseptic connector secured to the inlet of the agitator or the outlet of the agitator, and a second aseptic connector or weldable tube secured to the other of the inlet of the agitator or the outlet of agitator. The agitator may be any of the agitators disclosed herein. A fluid channel is defined through the first aseptic connector, the agitator, and the second aseptic connector or a weldable tube closed to an external environment and sterilized.

In another aspect of the present disclosure, an agitator system includes an agitator, a first weldable tube secured to the inlet of the agitator or the outlet of the agitator, and a second weldable tube or an aseptic connector secured to the other of the inlet of the agitator or the outlet of the agitator. The agitator may be any of the agitators disclosed herein. A fluid channel is defined through the weldable tube, the agitator, and the second weldable tube or the aseptic connector closed to an external environment and sterilized.

In another aspect of the present disclosure, a kit includes an agitator and packaging material encapsulating the agitator in an envelope thereof such that the agitator is sealed from an external environment. The agitator may be any of the agitators disclosed herein. The entire envelope and agitator may be sterilized. The kit includes a first aseptic connector secured to the inlet of the agitator or the outlet of the agitator. The kit may include a second aseptic connector or weldable tube secured to other of the inlet of the agitator or the outlet of the agitator. The kit may include a first weldable tube secured to the inlet of the agitator or the outlet of the agitator. The kit may comprise a second weldable tube or an aseptic connector secured to the other of the inlet of the agitator or the outlet of the agitator.

In another aspect of the present disclosure, an agitator includes a unitary body defining an inlet, an outlet, and the fluid channel that extends between the inlet and the outlet. The fluid channel has a disruptor section with a plurality of trituration points that are configured to agitate a fluid passing therethrough such that cells within the fluid are deagglomerated from one another.

In aspects, the disruptor section includes a plurality of chambers that are in fluid communication with one another. Each chamber of the plurality of chambers may have a maximum diameter that is different from each adjacent chamber such that the changes in the maximum diameter are configured to deagglomerate cells from one another. The disruptor section may include smooth transitions between the maximum diameters of the chambers.

In some aspects, a longitudinal length of the body is defined linearly between the inlet and the outlet. The disruptor section may wrap around within the body such that a flow length of the disruptor section is greater than the longitudinal length of the body. The disruptor section may be configured to generate the centrifugal force within the fluid flowing therethrough.

In certain aspects, the disruptor section is defined by chamber walls with a textured surface that is configured to promote shear within the fluid passing through the disruptor section. The disruptor section may be configured to deagglomerate T-cells from one another within a fluid.

In another aspect of the present disclosure, a fluid processing system includes a supply vessel, a discharge vessel, and an agitator. The agitator is disposed between the supply vessel and the discharge vessel. The agitator includes a unitary body that defines an inlet, an outlet, and the fluid channel extending between the inlet and the outlet. The fluid channel has a disruptor section with a plurality of chambers that are in fluid communication with one another. Each chamber of the plurality of chambers creates a trituration point in the disruptor section.

In aspects, each chamber of the plurality of chambers has a maximum diameter that is different from each of the adjacent chambers.

In some aspects, the system includes an inflow conduit that extends from the supply vessel to the inlet of the agitator such that the interior of the supply vessel is in fluid communication with the inlet via the inflow conduit. The system may include additional processing equipment disposed between the agitator and the discharge vessel. The additional processing equipment may include at least one of storing equipment, freezing equipment, or filtering equipment.

In another aspect of the present disclosure, a method of manufacturing an agitator includes forming a body of the agitator. The body of the agitator includes a fluid channel that extends between an inlet and an outlet of the body. The method includes purging residual material from within the fluid channel after forming the body. The method also includes curing the body of the agitator after purging residual material from within the fluid channel.

In aspects, forming the body of the agitator includes forming the agitator with additive manufacturing techniques. Purging the residual material from within the fluid channel may include passing a solvent through the fluid channel. Purging the residual material from within the fluid channel may include passing a grease through the fluid channel. Passing the grease through the fluid channel may include the grease being keratin oil or silicone oil. Passing the grease through the fluid channel may include the grease being Krytox or silicone. Forming the body may include forming a unitary body. Forming the body of the agitator may include forming a fluid channel having an inlet section, disruptor section, and an outlet section. The disruptor section may be disposed between the inlet section and the outlet section. The disruptor section may include a plurality of separate fluid subchannels that extend between the inlet section and the outlet section in a range of 5 five microns to 500 microns or in a range of 0.1 microns to 2 centimeters. The minimum diameter of a disrupter section of the fluid channel is in a range of 0.001 millimeters to 10 millimeters.

In some aspects, forming the body of the agitator includes the disruptor section having a flow length between the inlet section and the outlet section that is greater than a longitudinal length of the agitator from an inlet end to an outlet end thereof. Forming the body of the agitator may include the disruptor section spiraling about the circumference of an agitator portion of the agitator disposed between an inlet and an outlet end of the agitator.

In certain aspects, the method includes expelling the grease from the fluid channel after curing the body. The method may include sterilizing the agitator after curing the body.

In another aspect of the present disclosure, a method of deagglomerating cells within a fluid includes initiating a flow of fluid. The method includes flowing the fluid through an agitator such that the fluid flows through multiple trituration points within the agitator. Cells within the fluid are deagglomerated as the fluid flows through the agitator. The method may include collecting fluid from the agitator.

In aspects, the method may include further processing the fluid before collecting the fluid from the agitator and after flowing fluid through the agitator. Flowing the fluid through the agitator may include flowing the fluid through a fluid channel of the agitator that has an inlet section, a disruptor section, and an outlet section with the disruptor section being disposed between the inlet section and the outlet section. The disruptor section may have an average diameter in a range of 0.1 microns to 2 centimeters.

In some aspects, flowing the fluid through the agitator includes flowing the fluid through a fluid channel of the agitator having an inlet section, a disruptor section, and an outlet section. The disruptor section may be disposed between the inlet section and the outlet section. The disruptor section may have a minimum diameter in a range of 0.001 millimeters to 10 millimeters.

In certain aspects, the flowing the fluid through the agitator includes flowing the fluid through a fluid channel of the agitator having an inlet section, a disruptor section, and an outlet section. The disruptor section may be disposed between the inlet section and the outlet section. The disruptor section may have a maximum diameter and a minimum diameter. A ratio between the maximum diameter and the minimum diameter may be in a range of 2:1 to 100:1.

In certain aspects flowing fluid through the disruptor section includes flowing the fluid through a plurality of fluid subchannels of the disruptor section. The plurality of fluid subchannels being separate from one another between the inlet section and the outlet section. Flowing fluid through the disruptor section may include the fluid flowing through a length of the disruptor section such that the fluid flows a length greater than a longitudinal length of the agitator from the inlet to an outlet of the agitator. Flowing fluid through the disruptor section may include the fluid flowing circumferentially about an agitator portion of the agitator such that flowing fluid within the disruptor section includes imposing a circumferential force in the fluid.

In aspects, flowing the fluid through the agitator includes separating cells from microcarrier within the fluid. Initiating the flow of fluid may include activating a pump to flow the fluid.

Further, to the extent consistent, any of the embodiments or aspects described herein may be used in conjunction with any or all of the other embodiments or aspects described herein.

### BRIEF DESCRIPTION OF THE DRAWINGS

Various aspects of the present disclosure are described hereinbelow with reference to the drawings, which are not necessarily drawn to scale, which are incorporated in and constitute a part of this specification, wherein:

FIG. 1 is a perspective view of an agitator provided in accordance with the present disclosure;

FIG. 2 is a transverse cross-sectional view taken along the section line 2-2 of FIG. 1;

FIG. 3 is a longitudinal cross-sectional view taken along the section line 3-3 of FIG. 1;

FIG. 4 is a longitudinal cross-sectional view of another agitator provided in accordance with the present disclosure;

FIG. 5 is a longitudinal cross-sectional view of another agitator provided in accordance with the present disclosure;

FIG. 6 is a longitudinal cross-sectional view of another agitator provided in accordance with the present disclosure;

FIG. 7 is a longitudinal cross-sectional view of another agitator provided in accordance with the present disclosure;

FIG. 8 is a longitudinal cross-sectional view of another agitator provided in accordance with the present disclosure;

FIG. 9 is a longitudinal cross-sectional view of another agitator provided in accordance with the present disclosure;

FIG. 10 is a longitudinal cross-sectional view of another agitator provided in accordance with the present disclosure;

FIG. 11 is a longitudinal cross-sectional view of another agitator provided in accordance with the present disclosure;

FIG. 12 is a perspective view of another agitator provided in accordance with the present disclosure;

FIG. 13 is a flowchart of a method of manufacturing an agitator provided in accordance with the present disclosure;

FIG. 14 is a flowchart of a method of agglomerating cells within a fluid provided in accordance with the present disclosure;

FIG. 15 is a perspective view of a kit provided in accordance with the present disclosure including a closed aseptic system; and

FIG. 16 is a perspective view of another kit provided in accordance with the present disclosure including another closed aseptic system.

### DETAILED DESCRIPTION

The present disclosure will now be described more fully hereinafter with reference to example embodiments thereof with reference to the drawings in which like reference numerals designate identical or corresponding elements in each of the several views. These example embodiments are described so that this disclosure will be thorough and complete, and will fully convey the scope of the disclosure to those skilled in the art. Features from one embodiment or aspect can be combined with features from any other embodiment or aspect in any appropriate combination. For example, any individual or collective features of method aspects or embodiments can be applied to apparatus, product, or component aspects or embodiments and vice versa. The disclosure may be embodied in many different forms and should not be construed as limited to the embodiments set forth herein; rather, these embodiments are provided so that this disclosure will satisfy applicable legal requirements. As used in the specification and the appended claims, the singular forms “a,” “an,” “the,” and the like include plural referents unless the context clearly dictates otherwise. In addition, while reference may be made herein to quantitative measures, values, geometric relationships or the like, unless otherwise stated, any one or more if not all of these may be absolute or approximate to account for acceptable variations that may occur, such as those due to manufacturing or engineering tolerances or the like.

Throughout this description, the term “upstream” refers to the portion of the device or component thereof that is closer to input or inlet of the device or component and the term “downstream” refers to the portion of the device or component thereof that is closer to the output or outlet of the device or component.

The agitators disclosed herein may be used in flow thorough processes to disrupt, separate, or deagglomerate cells within a mixture to allow for further processing or harvesting of the cells. The agitators disclosed herein may improve consistency in processing over previous devices. The agitators may be used to separate cells from microcarriers. In some embodiments, the agitators may be used to alter rheological properties of suspensions within the fluid. The apparatuses and methods may be helpful in cell therapy, cell expansion, or the separation of microcarriers.

The agitators disclosed herein include a plurality of chambers that create trituration points within a fluid flow path. The trituration points may aid in the deagglomeration of cells or the separation of microcarriers within the fluid. The agitators herein are shown with a variety of connectors which should not been seen as limiting. The agitators disclosed below may include barbed fittings, threaded fittings (internally or externally threaded), Luer connectors, push-in fittings, press-fit fitting, crimp fitting, shielded connectors or caps, or other suitable fittings to allow the connection of tubes or other devices to the agitators disclosed herein. The fittings may be male or female connectors depending on the application. The fittings on the inlet end and the outlet end of the agitators may be the same or different from one another. The fittings for the agitators may be formed of metal, plastics, or combinations thereof.

Referring now to FIGS. 1-3, an agitator is disclosed in accordance with the present disclosure and is referred to generally as agitator 100. The agitator 100 has an inlet portion 110, an outlet portion 190, and an agitator portion 130 positioned between the inlet portion 110 and the outlet portion 190. The agitator 100 defines a flow channel 106 that extends from the inlet portion 110 to the outlet portion 190.

The inlet portion 110 defines an inlet 112 of the agitator 100 that defines an inlet section 116 of the fluid channel 106. The inlet portion 110 may be sustainably cylindrical in shape with a substantially constant outer diameter and a substantially constant inner diameter. In some embodiments, the inlet portion 110 includes a barb 114 on the outer surface that is configured to allow a fluid conduit to be slid over the inlet portion 110 while preventing or discouraging removal of the fluid conduit from over the barb 114. The fluid conduit secured over the inlet portion 110 may be considered an inflow fluid conduit.

In embodiments, the inlet portion 110 flows into an inlet cavity 120 at a downstream end of the inlet portion 110. The inlet cavity 120 may have the same diameter as the inlet section 116 or may have a different diameter. As shown, the inlet cavity 120 is substantially spherical in shape with a diameter larger than the inlet section 116. The inlet cavity 120 receives fluid from the inlet section 116 and provides fluid to one or more disruptor sections 136 of the fluid channel 106 within the agitator portion 130 of the agitator 100. Each disruptor section 136 may be considered a sub-channel of the fluid channel 106. As shown, the agitator portion 130 of the agitator 100 defines four separate disruptor sections 136 that extend from the inlet cavity 120 to an outlet cavity 180. The disruptor sections 136 will be described in detail below.

The outlet cavity 180 is positioned at an upstream end of the outlet portion 190. The outlet cavity 180 and the outlet portion 190 may be similar to the inflow cavity 120 and the inflow portion 110, respectively. For example, as shown, the outlet cavity 180 is substantially spherical in shape and receives fluid from each of the disruptor sections 136 and outputs the fluid received into a single outlet section 196 of the fluid channel 106 that extends from the outlet cavity 180 to the outlet 198 of the outlet portion 190. The outlet section 196 may have a constant diameter defined by the inner diameter of the outlet portion 190. The outlet portion 190 may have a substantially constant outer diameter and may include a barb 194 on an outer surface thereof that is configured to secure an outflow fluid conduit to the agitator 100. The inlet section 116 and the outlet section 196 may have the same diameter or different diameters.

As noted above, the agitator portion 130 defines one or more disruptor sections 136 that extend through the agitator portion 130 between the inlet cavity 120 and the outlet cavity 180. As shown, the agitator portion 130 defines four disruptor sections 136 between the inlet cavity 120 and the outlet cavity 180. Each disruptor section 136 may be substantially similar to the other disruptor sections 136 such that flow through each disruptor section 136 deagglomerates cells in a mixture in a similar manner to the other disruptor sections 136. The flow from the inlet cavity 120 may be equally divided between each of the disruptor sections 136 and then combined again in the outlet cavity 180. While the agitator portion 130 is shown with four disruptor sections 136, it is contemplated that an agitator portion 130 may have between 1 disruptor section and 128 disruptor sections, e.g., 1, 2, 4, 8, 16, 32, 64, or 128. In some embodiments, an agitator portion 130 may have more than 128 disruptor sections or multiple agitators may branch off of a single connection such that a single flow path may be divided between multiple agitators 100 and then recombined into a single flow path. The number of disruptor sections 136 may be determined by the amount of fluid being processed and the flow rate of the fluid. The disruptor sections 136 may be sized and configured based on a specific process. Each disruptor section 136 may have a mean diameter in a range of 0.1 micron to 2 centimeters, e.g., 1 millimeter to 5 millimeters or 2.5 millimeters to 10 millimeters. The term “mean diameter” refers to the average diameter of the respective disruptor section or a diameter of a connection section leading into or out of the respective disruptor section. Each disrupter section 136 may have a minimum diameter and a maximum diameter. The minimum diameter may be in a range of 0.001 millimeters to 10 millimeters. The ratio of maximum diameter to minimum diameter of each disrupter section may be in a range of 2:1 to 100:1. The change in diameter between chambers or within a chamber causes a pressure change and a change in velocity of a fluid flowing through the disrupter section. This change in pressure and/or velocity may create a shear in the fluid flowing through the disrupter section. The shear may deagglomerate the fluid flowing therethrough.

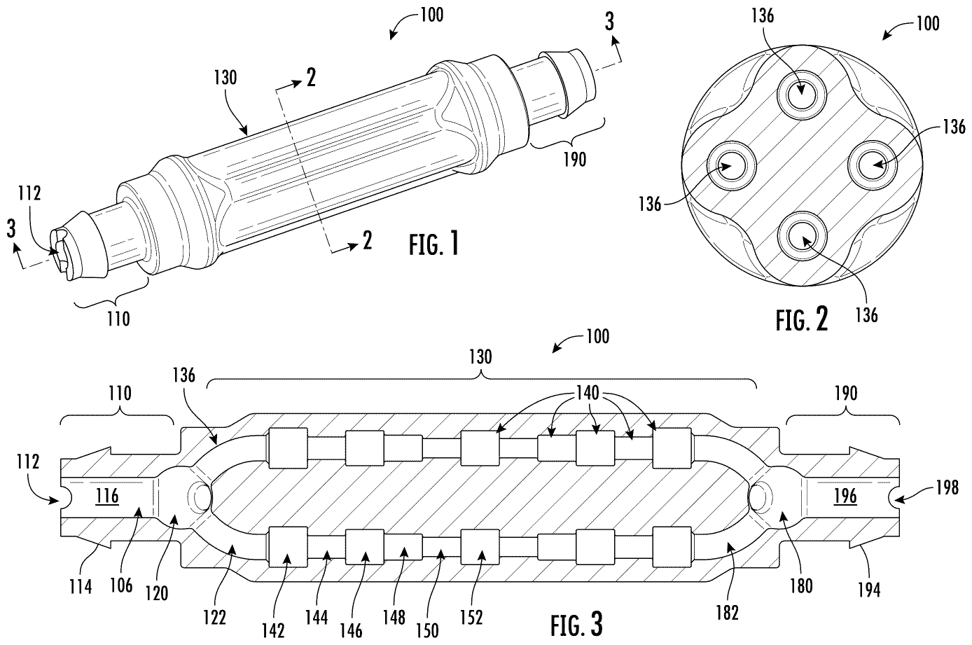

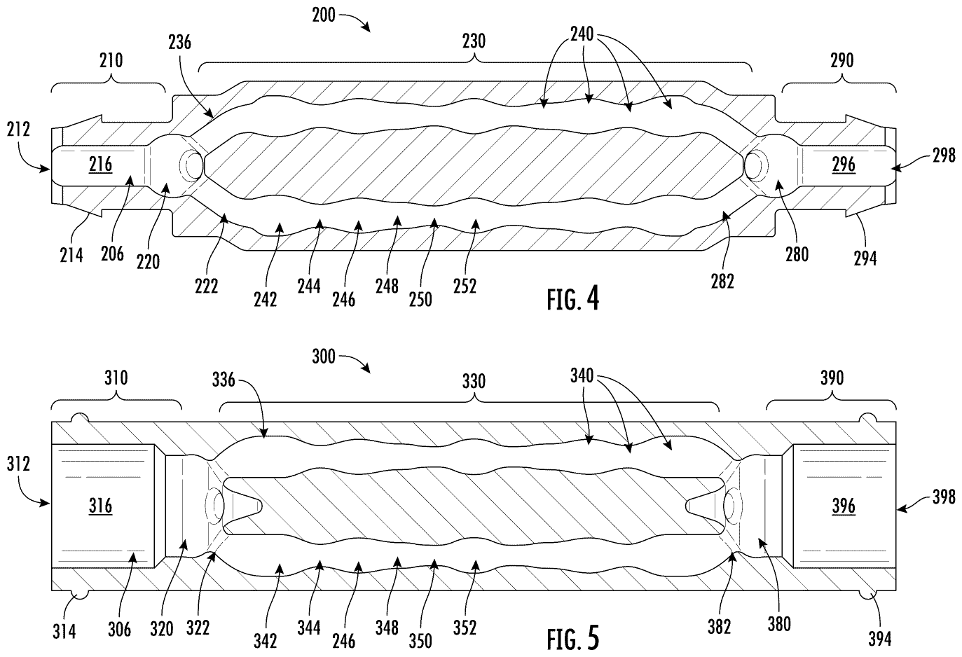

With particular reference to FIG. 3, only one of the disruptor sections 136 will be described for reasons of brevity with the other disruptor sections 136 being similar to the one that is described. The disruptor section 136 has an upstream connection section 122 that extends from the inlet cavity 120 and a downstream connection section 182 that extends to the outlet cavity 180. The connection sections 122, 182 provide flow into and out of the disruptor section 136. Each connection section 122, 182 may have a substantially constant diameter. The area of each connection section 122, 182 may be equal to a quarter of the area of the inlet section 116. The area of the connection portions may be determined by a simple equation of: Area of Connection Portion=Area of inlet fluid conduit/Number of Disruptor sections.

The disruptor section 136 between the upstream connection section 122 and the downstream connection section 182 is formed of a plurality of chambers 140. Each chamber 140 creating a change in pressure within a fluid flowing therethrough such that each chamber 140 can be considered a trituration point of the disruptor section 136. In some embodiments, each chamber 140 has a constant diameter that is different from the adjacent chambers 140. In certain embodiments, the chamber 140 has a changing diameter with the average diameter of the chamber 140 being different from adjacent chambers 140. In particular embodiments, adjacent chambers 140 have the same average diameter with the diameter of each chamber 140 changing along the length of the respective chamber 140 to cause a trituration point for the chamber 140. The trituration point created by the chambers 140 may cause deagglomeration of cells within a mixture as the mixture flows through the disruptor section 136. As shown, the disruptor section 136 has 11 chambers 140 or trituration points between the upstream connection section 122 and the downstream connection section 182. In some embodiments, a disruptor section 136 may have more or less than 11 chambers 140. The number of chambers 140 or trituration points for a particular disruptor section 136 may be in a range of 5 to 200, e.g., 5, 10, 15, or 20 chambers. In certain embodiments, each disruptor section 136 may have more than 200 chambers 140. As fluid flows downstream through the disruptor section 136, the trituration points created by the chambers 140 may cause pressure changes in the fluid or may change flow characteristics within the fluid such that cells within the fluid are deagglomerated as the fluid flows through the disruptor section 136. The changes in diameter between the chambers 140 may be abrupt or may be gradual. The changes in pressure or flow characteristics may be gentle such that damage to cells within the fluid is prevented or minimized.

As shown, the diameters of the chambers 140 are contestant with the maximum and average diameter of each chamber 140 being the same. Specifically, the diameter of the first chamber 142 is greater than the diameter of the upstream connection section 122, the diameter of the second chamber 144 is less than the diameter of the first chamber 142, the diameter of the third chamber 146 is greater than the diameter of the second chamber 144, the diameter of the fourth chamber 148 is less than the diameter of the third chamber 146, the diameter of the fifth chamber 150 is less than the diameter of the fourth chamber 148, and the diameter of the sixth chamber 152 is greater than the diameter of the fifth chamber 150. The diameters of the seventh, eighth, nineth, tenth, and eleventh chambers may follow the reverse order or be mirrored about the midpoint of the disruptor section 136. In some embodiments, the diameters of the seventh, eighth, nineth, tenth, and eleventh chambers may be different from or bear no resemblance to the diameters of the previous chambers. An example table for the diameters and areas of channels and the chambers

| Channel/Chamber | Inlet | Connector | 1 | 2 | 3 | 4 | 5 |
|---|---|---|---|---|---|---|---|
| Diameter (in) | 0.145 | 0.090 | 0.145 | 0.080 | 0.125 | 0.093 | 0.069 |
| Percent Change | | −38% | 61% | −45% | 56% | −26% | −26% |
| Ratio of Max/Min | | 8:5 | 9:5 | 9:5 | 11:7 | 4:3 | 4:3 |
| Area (in\hat{ }2) | 0.017 | 0.006 | 0.017 | 0.005 | 0.012 | 0.007 | 0.004 |
| Percent Change | | −61% | 160% | −70% | 144% | −45% | −45% |

| Channel/Chamber | 6 | 7 | 8 | 9 | 10 | 11 | Connector | Outlet |
|---|---|---|---|---|---|---|---|---|
| Diameter (in) | 0.125 | 0.069 | 0.093 | 0.125 | 0.080 | 0.145 | 0.090 | 0.145 |
| Percent Change | 81% | −45% | 35% | 34% | −36% | 81% | −38% | 61% |
| Ratio of Max/Min | 9:5 | 9:5 | 4:3 | 11:7 | 11:7 | 9:5 | 8:5 | 8:5 |
| Area (in\hat{ }2) | 0.012 | 0.004 | 0.007 | 0.012 | 0.005 | 0.017 | 0.006 | 0.017 |
| Percent Change | 228% | −70% | 82% | 81% | −59% | 229% | −61% | 160% |

As shown above, the change in diameter between adjacent channels or chambers may be in a range of −50 percent to 85 percent with a corresponding change in area being in a range of −70 percent to 250 percent. It will be appreciated that the values given above are for a particular example and the diameters and the percent changes between adjacent channels and chambers may be greater or less than described depending on cells in a fluid, the flow rate, the pressure, or other flow characteristics. The diameters and the percent changes between adjacent channels may be varied based on any of the flow characteristics. It should be noted that in this example the flow length of each chamber 140 may be equal to the flow length of each other chamber 140. In some embodiments, the flow length of the chambers 140 may vary from each chamber 140 to the next. In certain embodiments, the flow length of the chamber 140 may be determined by the diameter of the chamber 140. For example, the first chamber 142 may have a flow length that is less than the second chamber 144 when the diameter of the first chamber 142 is greater than the diameter of the second chamber 144. Likewise, the flow length of the second chamber 144 may be greater than the flow length of the third chamber 146 when the diameter of the third chamber 146 is greater than the diameter of the second chamber 144. Changing the flow length of respective chambers 140 may lessen a change in pressure between adjacent chambers 140 as the volume of the adjacent chambers 140 may be more equal to one another.

In embodiments, the surfaces defining the disruptor section 136 may aid in deagglomeration of cells within the fluid. For example, the surfaces defining the disruptor section 136 may have texture that increases or decreases shear within the fluid flowing therethrough. For example, the surfaces defining the disruptor section 136 may have a roughness to cause shear within the fluid flowing therethrough. In some embodiments, the surfaces defining the disruptor section 136 may have a sharkskin pattern to cause shear within the fluid flowing therethrough. In certain embodiments, one or more sections of the disruptor section 136 may have a smooth surface to decrease shear within the fluid flowing therethrough. In particular embodiments, the surface texture may vary through the disruptor section 136 to promote deagglomeration of cells in the fluid flowing therethrough. It is contemplated that it may be possible to have a disruptor section 136 with a constant diameter but with varying surface texture such that cells within a fluid are deagglomerated as the fluid flows through the disruptor section 136. Such a constant diameter disruptor section 136 may be more effective for very small diameter channels, e.g., disruptor sections having a diameter in a range of 1 micron to 1,000 microns, e.g., 5 microns to 500 microns.

The agitator 100 detailed above is configured to gently deagglomerate cells within a fluid flowing therethrough. The agitator 100 may detach or separate cells from microcarriers while minimizing or preventing damage to the cells. The agitator 100 detailed above may produce a suspension of single cells that can be harvested for further use. In some embodiments, cells can be separated from each other and/or microcarriers without the use of enzymes or with a reduced concentration of enzymes.

A variety of different types of cells can be separated and harvested according to embodiments of the invention. For example, separation and harvesting of adherent cells grown on microcarriers that can be used in seed train cell expansion for seeding larger bioreactors or for isolation of cells used in cell and gene therapy applications. Suitable cells include, but are not limited to CHO, BHK21, HEK293, Vero, MDCK, MDBK, A549, primary chondrocytes, primary liver, primary renal, bone marrow-derived mesenchymal stem/stromal cells, adipose-derived mesenchymal stem/stromal cells, embryonic stem cells, T-cells, and induced pluripotent stem cells. The agitators 100 may be used to separate a variety of microcarriers from cells. Microcarriers may be provided with a nominal size range which is specific for each product type. Microcarriers may be composed of multiple different materials including, but not limited to, rigid polymers, biodegradable substances (e.g., cellulose, fibrinogen, alginate or pectin), glass, or other materials.

With reference to FIG. 4, another agitator 200 is provided in accordance with the present disclosure. The agitator 200 is similar to the agitator 100 of FIGS. 1-3 with like elements represented with similar labels with the leading “1” of the element of agitator 100 with a leading “2” for the element of the agitator 200. For reasons of brevity, only the differences between the agitator 200 and the agitator 100 will be discussed below.

The disruptor sections 236 of the agitator 200 smoothly transitions between diameters of the channels 222, 282 and the chambers 240. As shown, the chambers 240 have an average diameter, a maximum diameter, and aa minimum diameter. The average diameter of adjacent chambers 240 maybe different to create trituration points between adjacent chambers. In some embodiments, the average diameter of adjacent chambers 240 may be the same with the maximum and minimum diameters of each chamber 240 causing a trituration point. The maximum or minimum diameters of the respective chamber 240 gradually increases or decreases in diameter to the subsequent chamber 240 to gradually transition diameters between chambers 240. The gradual transitions between the maximum diameters of the chambers 240 may maintain flow characteristics within the disruptor section 236. For example, the gradual transitions may maintain laminar flow within the disruptor section 236 as the fluid flows through the disruptor section 236. The changes in the diameter of the chambers 240 of the disruptor section 236 deagglomerate cells within fluid flowing through the disruptor section 236. The difference between the maximum and minimum diameter of each chamber 240 may be in a range of 4:3 to 100:1, e.g., 4:3, 3:2, 2:1, 5:2, 3:1. 4:1, 5:1, 10:1, 20:1, 40:1, 100:1. The change in diameter of each chamber 240 may causes a pressure change and a change in velocity of a fluid flowing through the disrupter section. This change in pressure and/or velocity may create a shear in the fluid flowing through the disrupter section. The shear may deagglomerate the fluid flowing therethrough

With reference to FIG. 5, another agitator 300 is provided in accordance with the present disclosure. The agitator 300 is similar to the agitator 200 of FIG. 4 with like elements represented with similar labels with the leading “2” of the element of agitator 200 with a leading “3” for the element of the agitator 300. For reasons of brevity, only the differences between the agitator 300 and the agitator 200 will be discussed below.

The outer surface of the agitator 300 may form a substantially cylindrical surface. The inlet portion 310 and the outlet portion 390 may include lugs 314, 394 respectively such that each of the inlet portion 310 and the outlet portion 390 are configured to receive a luer connector to fluidly couple a fluid conduit to the fluid channel 306 of the agitator 300. As shown, the inlet section 316 and the outlet section 396 may have a diameter greater than the inlet chamber 320 and the outlet chamber 380. Further, the disruptor sections 336 of the agitator 300 smoothly transition between the diameters of the chambers 340 to create a trituration point for each chamber 340. In some embodiments, the disruptor sections 336 may abruptly transition between the diameters of the chambers 340 in a similar manner to the disruptor sections 136 of the agitator 100 to create a trituration point for each chamber 340 as detailed above.

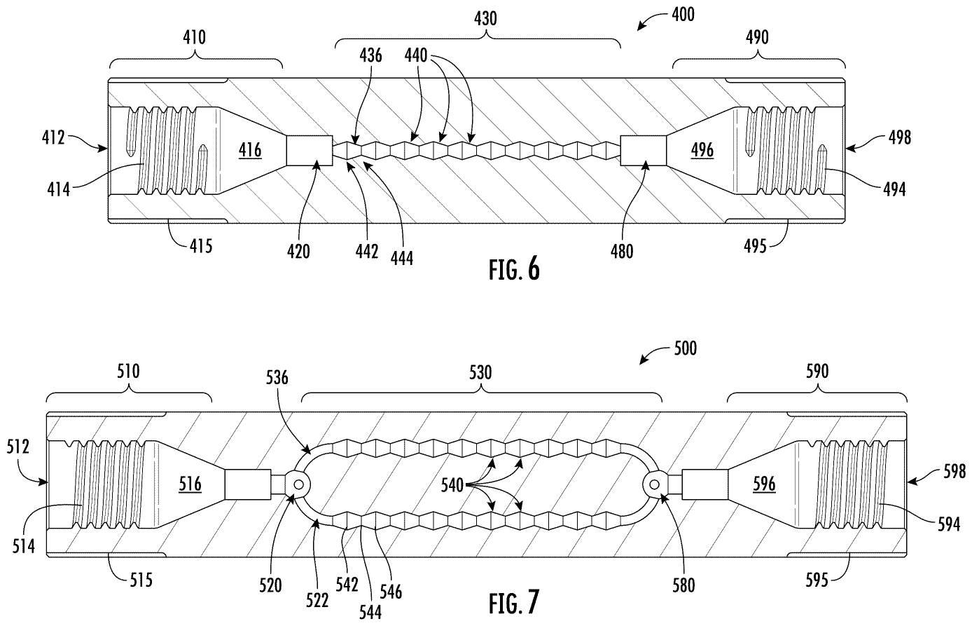

With reference to FIG. 6, another agitator 400 is provided in accordance with the present disclosure. The agitator 400 is similar to the agitator 100 of FIG. 1 with like elements represented with similar labels with the leading “1” of the element of agitator 100 with a leading “4” for the element of the agitator 400. For reasons of brevity, only the differences between the agitator 400 and the agitator 100 will be discussed below.

The outer surface of the agitator 400 may form a substantially cylindrical surface. The inlet portion 410 and the outlet portion 490 may include internal threading 414, 494 to receive a fluid conduit therein. The outer surface may define surface features 415 to aid in gripping the body of the agitator 400 to secure fluid conduits therein. As shown, the surface features 415 are flutes that extend longitudinally in the outer surface to allow for gripping of the body. In some embodiments, the surface features may be knurling, external fins, bumps, recess, textures, tabs, or other similar features to allow for a user to engage the outer surface.

Such a fluid conduit may include external threads that complement the internal threading 414, 494 to secure the respective fluid conduit to the agitator 400. The agitator 400 may be focused on smaller molecules or cells such as T-cells with the diameter of the disruptor section 436 varying in a range of 0.1 mm to 2 mm, e.g., 0.5 mm to 1 mm. In some embodiments, the disrupter section 436 has a minimum diameter of 500 microns and a maximum diameter of 1000 microns. In certain embodiments, the disrupter section 436 has a minimum diameter of 660 microns and a maximum diameter of 1120 microns. In particular embodiments, the ratio of the maximum diameter to the minimum diameter may be 2:1. While the decrease in diameter may lower a throughput of fluid through the agitator 400, the decrease in diameter may be necessary to create trituration points to deagglomerate smaller cells. In some embodiments, the agitator 400 may be considered a micro agitator. The transitions between the diameters of the chambers 440 may be linear, exponential, logarithmic, or other curved function. The transitions between the diameters may be gradual or abrupt to create trituration points.

As shown, the agitator 400 has a single disruptor section 436. In certain embodiments, the agitator 400 may have multiple disruptor sections 436.

For example, with reference to FIG. 7, another agitator 500 is provided in accordance with the present disclosure. The agitator 500 is similar to the agitator 400 of FIG. 6 with like elements represented with similar labels with the leading “4” of the element of agitator 400 with a leading “5” for the element of the agitator 500. For reasons of brevity, only the differences between the agitator 500 and the agitator 400 will be discussed below. The agitator 500 includes multiple disruptor sections 536 that extend between an inlet chamber 520 and an outlet chamber 580.

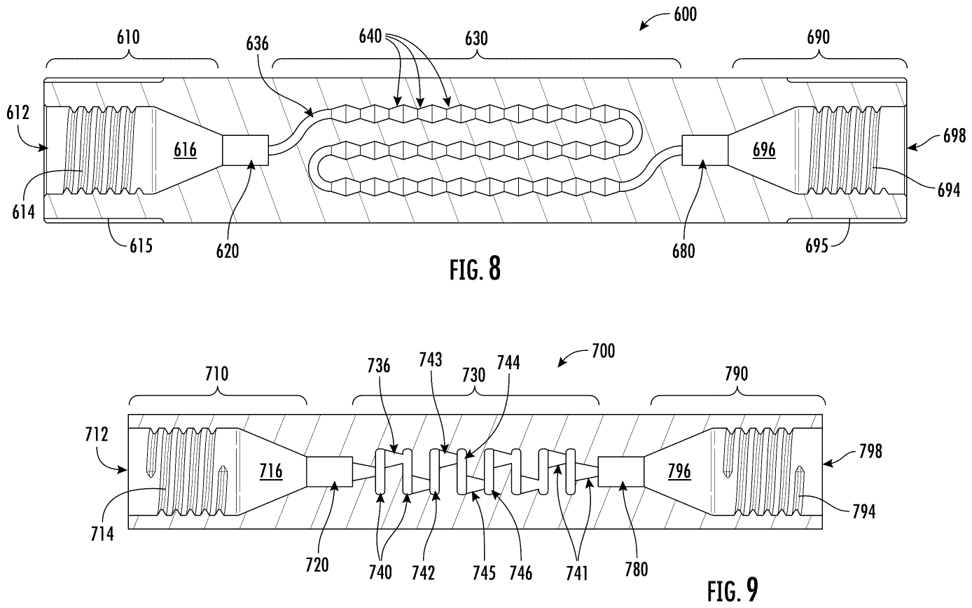

Referring now to FIG. 8, another agitator 600 is provided in accordance with the present disclosure. The agitator 600 is similar to the agitator 400 of FIG. 6 with like elements represented with similar labels with the leading “4” of the element of agitator 400 with a leading “6” for the element of the agitator 600. For reasons of brevity, only the differences between the agitator 600 and the agitator 400 will be discussed below.

The agitator 600 includes a single disruptor section 636 that wraps within the agitator portion 630 of the agitator 600. As shown, the disruptor section 636 has a total length that is substantially three times the length of the agitator portion 630. The extended length of the disruptor section 636 may allow for improved deagglomeration of cells within a fluid flowing through the disruptor section 636. The wrapping of the disruptor section 636 may allow for additional trituration points for a given length of an agitator. The wrapping of the disputer section 636 may allow for a decreased length or size of a given agitator. In certain embodiments, the agitator 600 may include multiple disruptor sections 636 that wrap within the agitator portion 630.

With reference to FIG. 9, another agitator 700 is provided in accordance with the present disclosure. The agitator 700 is similar to the agitator 400 of FIG. 6 with like elements represented with similar labels with the leading “4” of the element of agitator 400 with a leading “7” for the element of the agitator 700. For reasons of brevity, only the differences between the agitator 700 and the agitator 400 will be discussed below.

The disruptor section 736 of the agitator 700 includes a series of chambers 740 and funnels 741 between the chambers 740 to create trituration points in the disruptor section 736. For example, a first chamber 742 is in fluid communication with a second chamber 744 by a funnel 743 and the second chamber 744 is in fluid communication with a third chamber 746 by a funnel 745. The funnels 743, 745 may be offset from one another such that as fluid flows from one chamber through a subsequent chamber, e.g., from chamber 742 to chamber 744, the fluid must flow transversely within the chamber to exit the chamber. Further, as a fluid flows into a chamber, the fluid may flow directly into a wall defining the chamber to further disrupt cells within the fluid. For example, as fluid flows through a funnel, the fluid may be accelerated into a wall of the subsequent chamber. This acceleration may be considered splattering on the wall of the subsequent chamber.

With reference to FIG. 10, another agitator 800 is provided in accordance with the present disclosure. The agitator 800 is similar to the agitator 400 of FIG. 6 with like elements represented with similar labels with the leading “4” of the element of agitator 400 with a leading “8” for the element of the agitator 800. For reasons of brevity, only the differences between the agitator 800 and the agitator 400 will be discussed below. The inlet portion 810 and the outlet portion 890 may be configured to fluidly couple to a fluid conduit by a luer connector. To accommodate and couple with a luer connector, the outer surface of the agitator 800 may be formed in a manner to compliment a luer connector.

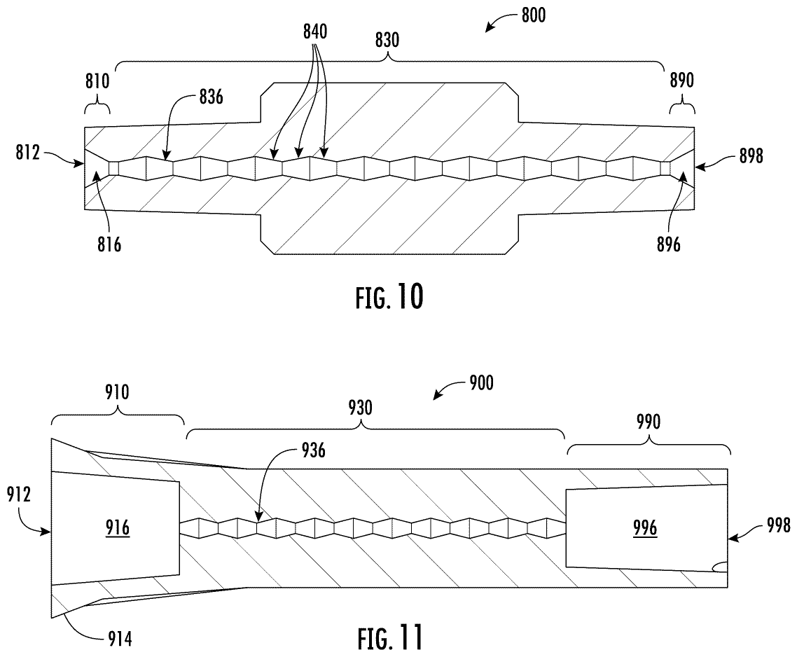

With reference to FIG. 11, another agitator 900 is provided in accordance with the present disclosure. The agitator 900 is similar to the agitator 400 of FIG. 6 with like elements represented with similar labels with the leading “4” of the element of agitator 400 with a leading “9” for the element of the agitator 900. For reasons of brevity, only the differences between the agitator 900 and the agitator 400 will be discussed below.

The inlet portion 910 may be configured to receive and fluidly couple to a pipette or a syringe. The pipette or syringe may be used to flow a fluid through the disruptor section 936 of the agitator 900 such that cells within the fluid are deagglomerated. This may be similar to the previous method of drawing and discharging a fluid from a pipette to deagglomerate cells within the fluid but may reduce a number of times that a fluid must be drawn and discharged. For example, the disruptor section 936 has at least 11 transitions or trituration points such that the number of times that a fluid must be drawn and discharged into a pipette may be reduced by at least a factor of 11 with the use of the agitator 900. The inlet portion 910 may include flares 914 that assist in gripping the inlet portion 910 to retain a pipette or syringe within the inlet portion 910.

The outlet portion 990 of the agitator 900 may be in fluid communication with a vial or a dish for receiving the fluid from a pipette. The outlet portion 990 may include threading or other features to grip a neck or secure a vial or other container to the outlet portion 990 to receive or provide a fluid that is drawn or discharged from a pipette or syringe.

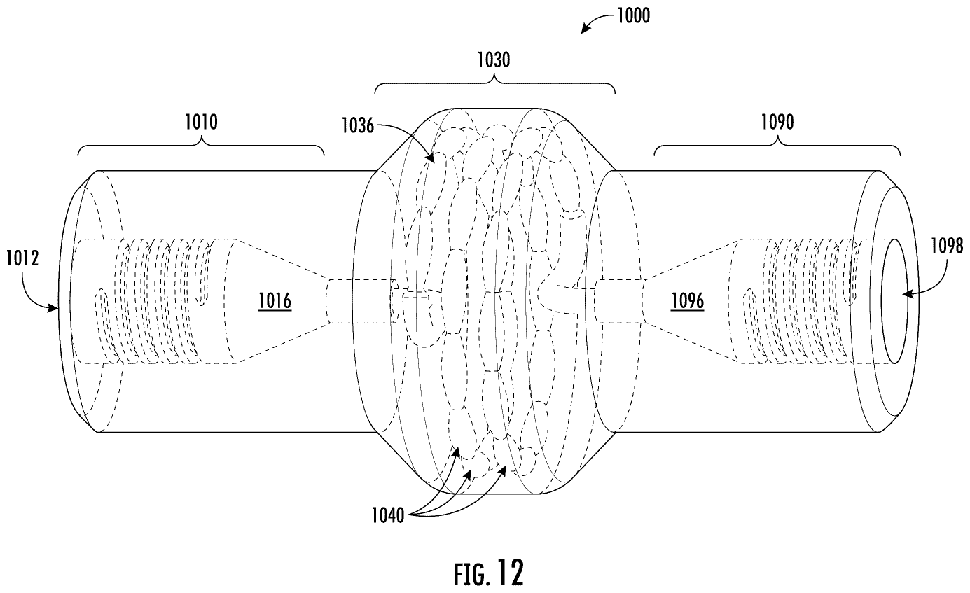

With reference to FIG. 12, another agitator 1000 is provided in accordance with the present disclosure. The agitator 1000 is similar to the agitator 600 of FIG. 8 with like elements represented with similar labels with the leading “6” of the element of agitator 600 with a leading “10” for the element of the agitator 1000. For reasons of brevity, only the differences between the agitator 1000 and the agitator 600 will be discussed below.

The agitator portion 1030 of the agitator 1000 has a diameter larger than the inlet portion 1010 and the outlet portion 1090. The disruptor section 1036 of the agitator 1000 extends into the disruptor section 1036 and spirals about the circumference of the agitator portion 1030 such that the disruptor section 1036 is coiled or wrapped within the agitator portion 1030 before flowing into the outlet portion 1090. The spiraling of the disruptor section 1036 may create a centrifugal force within fluid flowing through the disruptor section 1036. The centrifugal force may aid in deagglomeration of cells within the fluid. In addition, the spiraling of the disruptor section 1036 may increase the number of transitions or trituration points within the disruptor section 1036 for a given length of the agitator 1000.

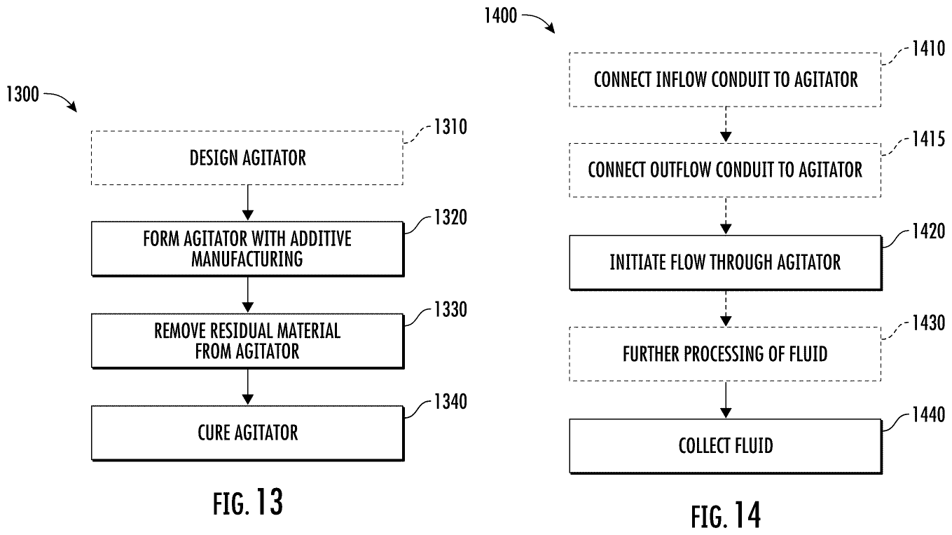

Referring now to FIG. 13, a method of manufacturing an agitator is provided in accordance with the present disclosure and is referred to generally as method 1300. Unless otherwise specified, the method 1300 will be described with reference to the agitator 100 of FIGS. 1-3. The method 1300 may include designing an agitator and in particular, disruptor sections of the agitator (Step 1310). The agitator may be designed in a software package that can deliver a three-dimensional model to an additive manufacturing system. The agitator and the disruptor sections thereof may be designed based on a particular fluid or process. For example, the disruptor sections may be designed to process a particular cell or to separate a particular microcarrier from a cell. The number of disruptor sections for a particular agitator may be determined by a desired flow rate of the agitator, the type of cells within the fluid, the type of fluid, degree of agglomeration, concentration of suspension, temperature, pressure, the size distribution of the microcarriers and other factors.

Once designed, the agitator is formed by an additive manufacturing system (Step 1320). To form the agitator, data is transferred to the additive manufacturing system. The additive manufacturing system may be any suitable manufacturing system including, but not limited to, a fused deposition modeling system, a stereolithography system, a selective laser sintering system, a digital light processing system, a sheet lamination system, or a metal binder jetting system. The agitator may be any of the agitators detailed herein. The agitator is formed as a monolithic unit and has a unitary body. The internal features such as the disruptor sections 136 are formed with the internal curves, transitions, and desired surface features. However, due to the size of some of the internal features, residual material from the forming process may be left within the internal features. To remove this residual material, a solvent may be passed through the agitator to remove the residual material from the internal features (Step 1330). In embodiments, the solvent may be tripropylene glycol monomethyl ether (TPM), isopropyl alcohol (IPA), ethyl alcohol (ETOH), ethyl acetate, hexamethyl disiloxane (HMDS), a low molecular weight siloxanes such as Dowsil® OS fluid from Dow Chemical Company, fluorocarbon compounds such as Fluorinert® fluid from 3M, or combinations thereof. In some embodiments, the solvent may be a grease that is passed through the agitator to remove the residual material from the internal features. The grease may be a fluorinated grease such as Krytox®, a high vacuum grease such as Molykote®, a silicone grease that is passed through the agitator such that the residual material is removed. In some embodiments, the grease may be a silicone compound having a low viscosity such that damage to the green or non-cured agitator is avoided before curing of the agitator. To pass the solvent through the agitator 100 a conduit may be connected to the inlet 112 or the outlet 198 and the solvent may be passed through the agitator 100 such that the solvent flows through the disruptor sections 136 to urge out any residual material disposed within the disruptor sections 136. After the residual material is removed from within the formed agitator, the formed agitator may be cured (Step 1340). The solvent used may not react or interfere with the curing process while post baking the agitator the solvent may be expelled using an organic solvent. After curing, the agitator may be sterilized (Step 1350). The agitator may be sterilized by being irradiated or autoclaved. In some embodiments, the agitator may be sterilized when in additional packaging. For example, the disputer may be in a sealed package and be irradiated through the sealed package.

Referring now to FIG. 14, a method of deagglomerating cells within a fluid is disclosed in accordance with the present disclosure and is referred to generally as method 1400. The method will be described with reference to the agitator 100 of FIGS. 1-3; however, the method may utilize any of the agitators described herein. The method 1400 may be used to deagglomerate cells within a fluid. The method 1400 may use a single agitator or a plurality of agitators in parallel to deagglomerate cells within a fluid. The method 1400 includes initiating flow of a fluid into an inlet 112 of the agitator 100 (Step 1420). The method 1400 may include connecting an inlet fluid conduit to the inlet 112 of the agitator 100 (Step 1410) such that a fluid source upstream of the agitator 100 is in fluid communication with the inlet 112 through the inlet fluid conduit. The fluid source may be a bioreactor, a vessel, a bag, or another container holding a fluid. The method 1400 may include connecting an outlet fluid conduit to the outlet 198 of the agitator 100 such that a vessel, container, or apparatus downstream of the agitator 100 is in fluid communication with the agitator 100 (Step 1415).

With the agitator 100 in fluid continuation with the supply vessel and a downstream vessel, container, or apparatus, flow of fluid through the agitator 100 is initiated (Step 1420). The flow of fluid may be initiated by activating a pump to flow fluid through the agitator 100. The pump may be a peristaltic pump with the inlet fluid conduit passing through the peristaltic pump. The flow of fluid may be gravity feed from the supply vessel into the inlet 112. In some embodiments, the flow of fluid is provided at a constant flow rate or a constant pressure into the inlet 112. As the fluid flows through the disruptor sections 136 of the agitator 100, cells within the fluid are deagglomerated or are separated from microcarriers. After the fluid flows through the agitator 100, the fluid may be further processed (Step 1430) or collected (Step 1440). For example, the fluid may be frozen for future use. In some embodiments, the agitator 100 may be used in a process to deagglomerate cells within a fluid such that the cells may be harvested or further processed.

Although the method steps are described in a specific order, it should be understood that other steps may be performed in between described steps, described steps may be adjusted so that they occur at slightly different times, or the described steps may occur in any order unless otherwise specified.

The devices contemplated here are used in the areas of bioprocessing where cells need to be deagglomerated to allow for further processing. Other fields of use could be in improving dispersions in suspensions. Yet other applications could be in the use of the device to change the rheological properties of suspensions through gentle agitation. The method of using the inventive device to process cell suspensions that are difficult to handle due to agglomeration can be helpful in cell therapy, cell expansion, and separation of microcarriers.

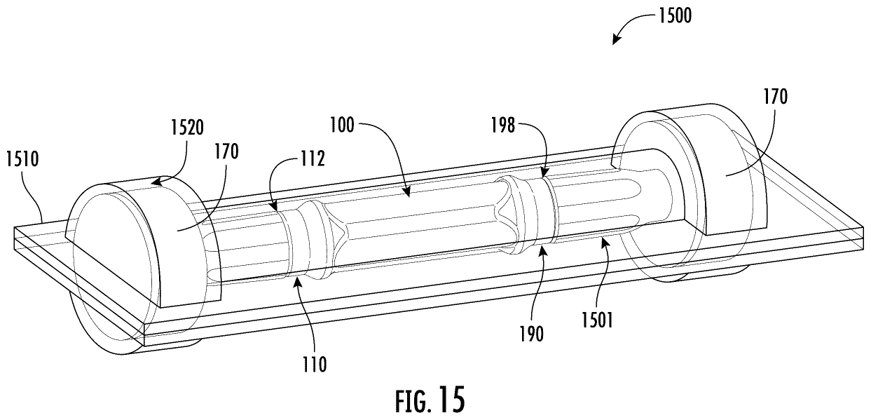

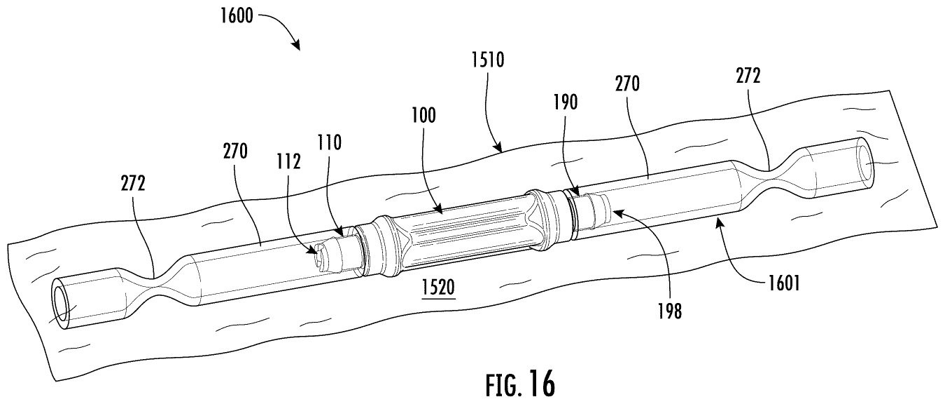

Referring now to FIGS. 15 and 16, kits are illustrated in accordance with embodiments of the present disclosure and are referred to generally as kit 1500 and kit 1600. The kits 1500, 1600 are shown including closed aseptic systems 1501, 1601 that each include a respective agitator, e.g., agitator 100 of FIGS. 1-3. While the aseptic systems 1501, 1601 are shown with agitator 100, any of the agitators disclosed herein may be used alone or in combination with other agitators in a kit similar to kits 1500, 1600.

With particular reference to FIG. 15, kit 1500 includes packaging material 1510 that forms an envelope 1520 that is sealed from an external environment. The packaging material 1510 may be configured to maintain a sterile or aseptic environment within the envelope 1520 such that the packaging material 1510 is capable of being used to seal and ship components within the envelope 1520 through nonsterile environment while maintaining an aseptic or sterile environment within the envelope 1520. The packaging material 1510 may be a plastic clamshell, a sealed plastic bag, a vacuum sealed plastic bag, or other suitable material. The agitator 100 is disposed and sealed within the envelope 1520 of the packaging material 1510. The agitator 100 and the packaging material 1510 may be sterilized with the agitator 100 sealed within the envelope 1520. In certain embodiments, the agitator 100 is sterilized before being sealed within the envelope 1520 and is sealed within the envelope 1520 in a sterile environment.

In embodiments, the agitator 100 may include aseptic connectors 170 secured to the inlet 112 and to the outlet 198 of the agitator 100. The aseptic connectors 170 allow for aseptic connection to the agitator 100 such that a fluid conduit may be aseptically placed in fluid communication with the fluid channel of the agitator 100. The aseptic connectors 170 being disposed within the envelope 1520. The aseptic connectors 170 may be sterilized before or after being sealed within the envelope 1520.

In some embodiments, the packaging material 1510 and the envelope 1520 may not be sterile. However, as the aseptic system 1501 is closed by having the aseptic connectors 170 on each end of the agitator 100, the fluid channel of the aseptic system 1501 is sterile and may be used in aseptic or sterile applications. The aseptic system 1501 may be sterilized after assembly or each element of the aseptic system 1501 may be individually sterilized and assembled in a clean room to maintain a sterile fluid channel therein.

With particular reference to FIG. 16, the aseptic system 1601 includes the agitator 100 with weldable tubing 270 secured to the inlet 112 and to the outlet 198 of the agitator 100. The weldable tubing 270 may include welded ends 272 to seal the fluid channel of the aseptic system 1601. The weldable tubing allows for aseptic connection to the agitator such that a fluid conduit or container may be aseptically placed in fluid communication with the fluid channel of the agitator 100. The weldable tubing 270 may be disposed within the envelope 1520. The aseptic connectors 170 may be sterilized before or after being sealed within the envelope 1520. In some embodiments, the agitator 100 may include an aseptic connector 170 on one of the inlet or the outlet and weldable tubing on the other of inlet and the outlet. In certain embodiments, the kits 1500, 1600 may include a container in sealed fluid communication downstream of the outlet 198.

In some embodiments, the packaging material 1510 and the envelope 1520 may not be sterile. However, as the aseptic system 1601 is closed by having the weldable tubing 270 sealed with the welds 272 on each end of the agitator 100, the fluid channel of the aseptic system 1601 is sterile and may be used in aseptic or sterile applications. The aseptic system 1601 may be sterilized after assembly or each element of the aseptic system 1601 may be individually sterilized and assembled in a clean room to maintain a sterile fluid channel therein.

While several embodiments of the disclosure have been shown in the drawings, it is not intended that the disclosure be limited thereto, as it is intended that the disclosure be as broad in scope as the art will allow and that the specification be read likewise. Any combination of the above embodiments is also envisioned and is within the scope of the appended claims. Therefore, the above description should not be construed as limiting, but merely as exemplifications of particular embodiments. Those skilled in the art will envision other modifications within the scope of the claims appended hereto.

What is claimed:

1. An agitator comprising:
a unitary body defining an inlet, an outlet, and a fluid channel extending between the inlet and the outlet, the fluid channel having a disruptor section, the disruptor section having a plurality of chambers in fluid communication with one another, each chamber creating a trituration point within the disruptor section.

2. The agitator according to claim 1, wherein the disruptor section has a minimum diameter and a maximum diameter, the minimum diameter in a range of 0.001 millimeters to 10 millimeters.

3. The agitator according to claim 1, wherein the unitary body defines an inlet chamber between the inlet and the fluid channel and an outlet chamber between the fluid channel and the outlet, the fluid channel including a plurality of fluid sub-channels with each fluid sub-channel having a respective disruptor section.

4. The agitator according to claim 1, wherein the plurality of chambers includes a first chamber, a second chamber, and a third chamber which are disposed in order with one another such that fluid flows downstream from the first chamber, directly into the second chamber, and directly into the third chamber, the first chamber having a first maximum diameter, the second chamber having a second maximum diameter that is less than the first maximum diameter, the third chamber having a third maximum diameter that is greater than the second maximum diameter.

5. The agitator according to claim 4, wherein the third maximum diameter is greater than the first maximum diameter.

6. The agitator according to claim 1, wherein the disruptor section includes smooth transitions between the maximum diameters of the chambers.

7. The agitator according to claim 1, wherein a longitudinal length of the body is defined linearly between the inlet and the outlet, the disruptor section wrapping within the body such that a flow length of the disputer section is greater than the longitudinal length of the body.

8. The agitator according to claim 7, wherein the flow length of the disruptor section is greater than twice the longitudinal length of the body.

9. The agitator according to claim 7, wherein the disruptor section is configured to generate a centrifugal force within the fluid flowing therethrough.

10. The agitator according to claim 1, wherein the disruptor section is defined by chamber walls, the chamber walls having a textured surface to promote shear within the fluid passing through the disruptor section.

11. The agitator according to claim 1, wherein the disruptor section is configured to deagglomerate cells from one another within a fluid as the fluid flows through the disruptor section.

12. The agitator according to claim 1, wherein the disruptor section is configured to separate cells from microcarriers within a fluid as a fluid flows through the disruptor section.

13. An agitator system comprising:
an agitator according to claim 1;
a first aseptic connector secured to the inlet of the agitator or the outlet of the agitator; and
a second aseptic connector or weldable tube secured to the other of the inlet of the agitator or the outlet of the agitator, a fluid channel defined through the first aseptic connector, the agitator, and the second aseptic connector or a weldable tube closed to an external environment and sterilized.

14. An agitator system comprising:
an agitator according to claim 1;
a first weldable tube secured to the inlet of the agitator or the outlet of the agitator; and
a second weldable tube or an aseptic connector secured to the other of the inlet of the agitator or the outlet of the agitator, a fluid channel defined through the weldable tube, the agitator, and the second weldable tube or the aseptic connector closed to an external environment and sterilized.

15. An agitator comprising:
a unitary body defining an inlet, an outlet, and a fluid channel extending between the inlet and the outlet, the fluid channel having a disruptor section, the disruptor section having a plurality of trituration points configured to agitate a fluid passing therethrough such that cells within the fluid are deagglomerated from one another.

16. The agitator according to claim 15, wherein the disruptor section includes a plurality of chambers that are in fluid communication with one another, each chamber of the plurality of chambers having a maximum diameter that is different from each of the adjacent chambers such that the changes in the maximum diameter are configured to deagglomerate cells from one another.

17. The agitator according to claim 15, wherein the disruptor section includes smooth transitions between the maximum diameters of the chambers.

18. The agitator according to claim 15, wherein the disruptor section is configured to deagglomerate T-cells from one another within a fluid.

19. A fluid processing system comprising:
a supply vessel;
a discharge vessel; and
an agitator disposed between the supply vessel and the discharge vessel, the agitator comprising a unitary body defining an inlet, an outlet, and a fluid channel extending between the inlet and the outlet, the fluid channel having a disruptor section, the disruptor section having a plurality of chambers that are in fluid communication with one another, each chamber of the plurality of chambers creates a trituration point in the disruptor section.

20. The system according to claim 19, wherein each chamber of the plurality of chambers has a maximum diameter that is different from each of the adjacent chambers.
